(19) United States
(12) Patent Application Publication
(10) Pub. No.: US 2009/0156853 A1
(43) Pub. Date: Jun. 18, 2009

(54) Process for industrially producing dialkyl carbonate and diol

(75) Inventors: Shinsuke Fukuoka, Tokyo (JP); Hironori Miyaji, Tokyo (JP); Hiroshi Hachiya, Tokyo (JP); Kazuhiko Matsuzaki, Tokyo (JP)

Correspondence Address: BIRCH STEWART KOLASCH & BIRCH, PO BOX 747, FALLS CHURCH, VA, 22040-0747, US

(73) Assignee: Asahi Kasei chemcials corporation, Chiyoda-ku Tokyo (JP)

(21) Appl. No.: 11/991,250

(22) PCT Filed: Dec. 6, 2006
(86) PCT No.: PCT/JP2006/324351

§ 371 Date: Oct. 23, 2008

(30) Foreign Application Priority Data
Dec. 13, 2005 (JP) 2005 358531

(51) Int. Cl. C07C 68/06

(52) U.S. Cl. 558/277

(57) ABSTRACT

It is an object of the present invention to provide a specific process that enables a dialkyl carbonate and a diol to be produced on an industrial scale of not less than 2 ton/hr and not less than 1.3 ton/hr respectively with high selectivity and high productivity stably for a prolonged period of time through a reactive distillation system of taking a cyclic carbonate and an aliphatic monohydric alcohol as starting materials, continuously feeding the starting materials into a continuous multi-stage distillation column in which a catalyst is present, and carrying out reaction and distillation simultaneously in the column. Although there have been many proposals regarding processes for the production of the dialkyl carbonate and the diol through the reactive distillation method, these have all been on a small scale and short operating time laboratory level, and there have been no disclosures whatsoever on a specific process or apparatus enabling mass production on an industrial scale. According to the present invention, there is provided a production process using the continuous multi-stage distillation column having a specified structure that enables the dialkyl carbonate and the diol to be produced on an industrial scale of not less than 2 ton/hr and not less than 1.3 ton/hr respectively each with a high selectivity of not less than 95%, preferably not less than 97%, more preferably not less than 99%, stably for not less than 1000 hours, preferably not less than 3000 hours, more preferably not less than 5000 hours, wherein the starting materials are introduced into specified stages of the distillation column, and the starting material aliphatic monohydric alcohol has a specified composition. As a result, the above object can be attained easily.

### TECHNICAL FIELD

The present invention relates to an industrial process for the production of a dialkyl carbonate and a diol. More particularly, the present invention relates to a process for industrially producing large amounts of the dialkyl carbonate and the diol stably for a prolonged period of time through a reactive distillation system of taking a cyclic carbonate and an aliphatic monohydric alcohol as starting materials, continuously feeding the starting materials into a continuous multi-stage distillation column in which a catalyst is present, and carrying out reaction and distillation simultaneously in the column.

### BACKGROUND ART

Several processes for the production of a dialkyl carbonate and a diol from a reaction between a cyclic carbonate and an aliphatic monohydric alcohol have been proposed, but most of these proposals relate to a catalyst. As reaction systems, four systems have been proposed hitherto. These four reaction systems are used in a process for the production of dimethyl carbonate and ethylene glycol from ethylene carbonate and methanol, which is the most typical reaction example.

A first system is a completely batch reaction system in which ethylene carbonate, methanol and a catalyst are put into an autoclave, which is a batch reaction vessel, and reaction is carried out by holding for a predetermined reaction time under an applied temperature at a reaction temperature above the boiling point of methanol (see, for example, Patent Document 1: U.S. Pat. No. 3,642,858, Patent Document 2: Japanese Patent Application Laid-Open No. S54-48715 (corresponding to U.S. Pat. No. 4,181,676), Patent Document 5: Japanese Patent Application Laid-Open No. S54-63023, Patent Document 6: Japanese Patent Application Laid-Open No. S54-148726).

A second system is a system that uses an apparatus in which a distillation column is provided on top of a reaction vessel; ethylene carbonate, methanol and a catalyst are put into the reaction vessel, and reaction is made to proceed by heating to a predetermined temperature. With this system, to make up for methanol distilled off through azeotropy with the produced dimethyl carbonate, methanol is added to the reaction vessel continuously or in batches, but in any event with this system the reaction proceeds only in the reaction vessel, which is batch type, in which the catalyst, ethylene carbonate and methanol are present. The reaction is thus batch type, the reaction being carried out using a large excess of methanol under reflux taking a long time in a range of from 3 to over 20 hours (see, for example, Patent Document 3: Japanese Patent Application Laid-Open No. S51-122025 (corresponding to U.S. Pat. No. 4,062,884), Patent Document 4: Japanese Patent Application Laid-Open No. S54-48716 (corresponding to U.S. Pat. No. 4,307,032), Patent Document 11: U.S. Pat. No. 3,803,201).

A third system is a continuous reaction system in which a mixed solution of ethylene carbonate and methanol is continuously fed into a tubular reactor maintained at a predetermined reaction temperature, and a reaction mixture containing unreacted ethylene carbonate and methanol and product dimethyl carbonate and ethylene glycol is continuously withdrawn in a liquid form from an outlet on the other side. Either of two processes is used depending on the form of the catalyst used. That is, there are a process in which a homogeneous catalyst is used, and is passed through the tubular reactor together with the mixed solution of ethylene carbonate and methanol, and then after the reaction the catalyst is separated out from the reaction mixture (see, for example, Patent Document 7: Japanese Patent Application Laid-Open No. S63-41432 (corresponding to U.S. Pat. No. 4,661,609), Patent Document 10: U.S. Pat. No. 4,734,518), and a process in which a heterogeneous catalyst fixed inside the tubular reactor is used (see, for example, Patent Document 8: Japanese Patent Application Laid-Open No. S63-238043, Patent Document 9: Japanese Patent Application Laid-Open No. S64-31737 (corresponding to U.S. Pat. No. 4,691,041)). The reaction producing dimethyl carbonate and ethylene glycol through reaction between ethylene carbonate and methanol is an equilibrium reaction, and hence with this continuous flow reaction system using a tubular reactor, it is impossible to make the ethylene carbonate conversion higher than the equilibrium conversion determined by the composition ratio put in and the reaction temperature. For example, according to Example 1 in Patent Document 7 (Japanese Patent Application Laid-Open No. S63-41432 (corresponding to U.S. Pat. No. 4,661,609)), for a flow reaction at 130° C. using a starting material put in with a molar ratio of methanol/ethylene carbonate=4/1, the ethylene carbonate conversion is 25%. This means that a large amount of unreacted ethylene carbonate and methanol remaining in the reaction mixture must be separated out, recovered, and recirculated back into the reactor, and in actual fact, with the process of Patent Document 9 (Japanese Patent Application Laid-Open No. S64-31737 (corresponding to U.S. Pat. No. 4,691,041)), much equipment is used for such separation, purification, recovery, and recirculation.

A fourth system is a reactive distillation system first disclosed by the present inventors (see, for example, Patent Document 12: Japanese Patent Application Laid-Open No. H4-198141, Patent Document 13: Japanese Patent Application Laid-Open No. H4-230243, Patent Document 14: Japanese Patent Application Laid-Open No. H9-176061, Patent Document 15: Japanese Patent Application Laid-Open No. H9-183744, Patent Document 16: Japanese Patent Application Laid-Open No. H9-194435, Patent Document 17: International Publication No. WO97/23445 (corresponding to European Patent No. 0889025, U.S. Pat. No. 5,847,189), Patent Document 18: International Publication No. WO99/64382 (corresponding to European Patent No. 1086940, U.S. Pat. No. 6,346,638), Patent Document 19: International Publication No. WO00/51954 (corresponding to European Patent No. 1174406, U.S. Pat. No. 6,479,689), Patent Document 20: Japanese Patent Application Laid-Open No. 2002-308804, Patent Document 21: Japanese Patent Application Laid-Open No. 2004-131394), that is a continuous production process in which ethylene carbonate and methanol are each continuously fed into a multi-stage distillation column, and reaction is carried out in the presence of a catalyst in a plurality of stages in the distillation column, while the product dimethyl carbonate and ethylene glycol are separated off. Patent applications in which such a reactive distillation system is used have subsequently been filed by other companies (see, for example, Patent Document 22: Japanese Patent Application Laid-Open No. H5-213830 (corresponding to European Patent No. 0530615, U.S. Pat. No. 5,231,212), Patent Document 23: Japanese Patent Application Laid-Open No. H6-9507 (corresponding to European Patent No. 0569812, U.S. Pat. No. 5,359,118), Patent Document 24: Japanese Patent Application Laid-Open No. 2003-119168 (corresponding to International Publication No. WO03/006418), Patent Document 25: Japanese Patent Application Laid-Open No. 2003-300936, Patent Document 26: Japanese Patent Application Laid-Open No. 2003-342209).

In this way, the processes proposed hitherto for producing a dialkyl carbonate and a diol from a cyclic carbonate and an aliphatic monohydric alcohol are the four systems:

(1) a completely batch reaction system;
(2) a batch reaction system using a reaction vessel having a distillation column provided on top thereof;
(3) a flowing liquid reaction system using a tubular reactor; and
(4) a reactive distillation system.

However, there have been problems with these as follows.

In the case of (1) and (3), the upper limit of the cyclic carbonate conversion is determined by the composition put in and the temperature, and hence the reaction cannot be carried out to completion, and thus the reaction ratio is low. Moreover, in the case of (2), to make the cyclic carbonate conversion high, the produced dialkyl carbonate must be distilled off using a very large amount of the aliphatic monohydric alcohol, and a long reaction time is required. In the case of (4), the reaction can be made to proceed with a higher conversion than with (1), (2) or (3). However, processes of (4) proposed hitherto have related to producing the dialkyl carbonate and the diol either in small amounts or for a short period of time, and have not related to carrying out the production on an industrial scale stably for a prolonged period of time. That is, these processes have not attained the object of producing a dialkyl carbonate continuously in a large amount (e.g. not less than 2 ton/hr) stably for a prolonged period of time (e.g. not less than 1000 hours, preferably not less than 3000 hours, more preferably not less than 5000 hours).

For example, the maximum values of the height (H: cm), diameter (D: cm), and number of stages (n) of the reactive distillation column, the produced amount P (kg/hr) of dimethyl carbonate, and the continuous production time T (hr) in examples disclosed for the production of dimethyl carbonate (DMC) and ethylene glycol (EG) from ethylene carbonate and methanol are as in Table 1.

TABLE 1

| PATENT | | | | | |
|---|---|---|---|---|---|
| DOCUMENT | H: cm | D: cm | NO. STAGES: n | P: kg/hr | T: hr |
| 12 | 100 | 2 | 30 | 0.106 | 400 |
| 15 | 160 | 5 | 40 | 0.427 | NOTE 5 |
| 16 | 160 | 5 | 40 | 0.473 | NOTE 5 |
| 18 | 200 | 4 | PACKING COLUMN (Dixon) | 0.932 | NOTE 5 |
| 19 | NOTE 1 | 5 | 60 | 0.275 | NOTE 5 |
| 20 | NOTE 1 | 5 | 60 | 0.258 | NOTE 5 |
| 21 | NOTE 1 | 5 | 60 | 0.258 | NOTE 5 |
| 22 | 250 | 3 | PACKING COLUMN (Raschig) | 0.392 | NOTE 5 |
| 23 | NOTE 2 | NOTE 2 | NOTE 2 | 0.532 | NOTE 5 |
| 24 | NOTE 3 | NOTE 3 | 42 | NOTE 4 | NOTE 5 |
| 25 | NOTE 3 | NOTE 3 | 30 | 3750 | NOTE 5 |
| 26 | 200 | 15 | PACKING COLUMN (BX) | 0.313 | NOTE 5 |

NOTE 1: OLDERSHAW DISTILLATION COLUMN.
NOTE 2: NO DESCRIPTION WHATSOEVER DEFINING DISTILLATION COLUMN.
NOTE 3: ONLY DESCRIPTION DEFINING DISTILLATION COLUMN IS NUMBER OF STAGES.
NOTE 4: NO DESCRIPTION WHATSOEVER OF PRODUCED AMOUNT.
NOTE 5: NO DESCRIPTION WHATSOEVER REGARDING STABLE PRODUCTION FOR PROLONGED PERIOD OF TIME.

In Patent Document 25 (Japanese Patent Application Laid-Open No. 2003-300936) (paragraph 0060), it is stated “The present example uses the same process flow as for the preferred mode shown in FIG. 1 described above, and was carried out with the object of operating a commercial scale apparatus for producing dimethyl carbonate and ethylene glycol through transesterification by a catalytic conversion reaction between ethylene carbonate and methanol. Note that the following numerical values in the present example can be adequately used in the operation of an actual apparatus”, and as that example it is stated that 3750 kg/hr of dimethyl carbonate was specifically produced. The scale described in that example corresponds to an annual production of 30,000 tons or more, and hence this implies that at the time of the filing of the patent application for Patent Document 25 (Japanese Patent Application Laid-Open No. 2003-300936) (Apr. 9, 2002), operation of the world's first large scale commercial plant using this process had been carried out. However, even at the time of filing the present application, there is not the above fact at all. Moreover, in the example of Patent Document 25 (Japanese Patent Application Laid-Open No. 2003-300936), exactly the same value as the theoretically calculated value is stated for the amount of dimethyl carbonate produced, but the yield for ethylene glycol is approximately 85.6%, and the selectivity is approximately 88.4%, and hence it cannot really be said that a high yield and high selectivity have been attained. In particular, the low selectivity indicates that this process has a fatal drawback as an industrial production process. (Note also that Patent Document 25 (Japanese Patent Application Laid-Open No. 2003-300936) was deemed to have been withdrawn on Jul. 26, 2005 due to examination not having been requested).

With the reactive distillation method, there are very many causes of fluctuation such as composition variation due to reaction and composition variation due to distillation in the distillation column, and temperature variation and pressure variation in the column, and hence continuing stable operation for a prolonged period of time is accompanied by many difficulties, and in particular these difficulties are further increased in the case of handling large amounts. To continue mass production of the dialkyl carbonate and the diol using the reactive distillation method stably for a prolonged period of time while maintaining high yields and high selectivities for the dialkyl carbonate and the diol, the reactive distillation apparatus must be cleverly devised. However, the only description of continuous stable production for a prolonged period of time with the reactive distillation method proposed hitherto has been the 200 to 400 hours in Patent Document 12 (Japanese Patent Application Laid-Open No. H4-198141) and Patent Document 13 (Japanese Patent Application Laid-Open No. H4-230243).

### DISCLOSURE OF INVENTION

### Problems to be Solved by the Invention

It is an object of the present invention to provide, for the case of producing the dialkyl carbonate and the diol industrially in large amounts (e.g. not less than 2 ton 1 hr for the dialkyl carbonate, and not less than 1.3 ton 1 hr for the diol) through a reactive distillation system of taking the cyclic carbonate and the aliphatic monohydric alcohol as starting materials, continuously feeding the starting materials into the continuous multi-stage distillation column in which a catalyst is present, and carrying out reaction and distillation simultaneously in the column, a specific process in which the dialkyl carbonate and the diol can be produced with high selectivity and high productivity stably for a prolonged period of time (e.g. not less than 1000 hours, preferably not less than 3000 hours, more preferably not less than 5000 hours).

### Means for Solving the Problems

Since the present inventors first disclosed a process for continuously producing the dialkyl carbonate and the diol using the continuous multi-stage distillation column, there have been many proposals regarding improving this process. However, these have been on a small scale and short operating time laboratory level, and there have been no disclosures whatsoever on a specific process or apparatus enabling mass production on an industrial scale stably for a prolonged period of time based on findings obtained through actual implementation. The present inventors have thus carried out studies aimed at discovering a specific process enabling the dialkyl carbonate and the diol to be produced on an industrial scale of, for example, not less than 2 ton/hr for the dialkyl carbonate and not less than 1.3 ton/hr for the diol stably for a prolonged period of time with high selectivity and high productivity

That is, the present invention provides:

1. an industrial process for the production of a dialkyl carbonate and a diol in which the dialkyl carbonate and the diol are continuously produced through a reactive distillation system of taking a cyclic carbonate and an aliphatic monohydric alcohol as starting materials, continuously feeding the starting materials into a continuous multi-stage distillation column in which a catalyst is present, carrying out reaction and distillation simultaneously in said column, continuously withdrawing a low boiling point reaction mixture containing the produced dialkyl carbonate from an upper portion of the column in a gaseous form, and continuously withdrawing a high boiling point reaction mixture containing the diol from a lower portion of the column in a liquid form, wherein:

(a) said continuous multi-stage distillation column comprises a tray column type distillation column having a cylindrical trunk portion having a length L (cm) and an inside diameter D (cm) and having a structure having thereinside an internal with a number of stages n, said internal being a tray having a plurality of holes, and further having a gas outlet having an inside diameter d₁ (cm) at a top of the column or in an upper portion of the column near to the top, a liquid outlet having an inside diameter d₂ (cm) at a bottom of the column or in a lower portion of the column near to the bottom, at least one first inlet provided in the upper portion and/or a middle portion of the column below said gas outlet, and at least one second inlet provided in the middle portion and/or the lower portion of the column above said liquid outlet, wherein:

(1) the length L (cm) satisfies the formula (1);

2100≦L≦8000 (1),

(2) the inside diameter D (cm) of the column satisfies the formula (2);

180≦D≦2000 (2),

(3) a ratio of the length L (cm) to the inside diameter D (cm) of the column satisfies the formula (3);

4≦L/D≦40 (3),

(4) the number of stages n satisfies the formula (4);

10≦n≦120 (4),

(5) a ratio of the inside diameter D (cm) of the column to the inside diameter d₁ (cm) of the gas outlet satisfies the formula (5)

3≦D/d₁≦20 (5), and

(6) a ratio of the inside diameter D (cm) of the column to the inside diameter d₂ (cm) of the liquid outlet satisfies the formula (6);

5≦D/d₂≦30 (6);

(b) the starting material cyclic carbonate is continuously introduced into said continuous multi-stage distillation column from at least one said first inlet provided between the 3^{rd} stage from the top of said continuous multi-stage distillation column and the (n/3)^{th} stage from the top of said continuous multi-stage distillation column;

(c) the starting material aliphatic monohydric alcohol contains in a range of from 1 to 15% by weight of the dialkyl carbonate; and

(d) the starting material aliphatic monohydric alcohol is continuously introduced into said continuous multi-stage distillation column from at least one said second inlet provided between the (n/3)^{th} stage from the top of said continuous multi-stage distillation column and the (2n/3)^{th} stage from the top of said continuous multi-stage distillation column,

2. the process according to item 1, wherein a produced amount of the dialkyl carbonate is not less than 2 ton/hr,
3. the process according to item 1 or 2, wherein a produced amount of the diol is not less than 1.3 ton/hr,
4. the process according to any one of items 1 to 3, wherein said d₁ and said d₂ satisfy the formula (7);

1≦d₁/d₂≦5 (7),

5. the process according to any one of items 1 to 4, wherein L, D, L/D, n, D/d₁, and D/d₂ for said continuous multi-stage distillation column satisfy respectively 2300≦L≦6000, 200≦D≦1000, 5≦L/D≦30, 30≦n≦100, 4≦D/d₁≦15, and 7≦D/d₂≦25,
6. the process according to any one of items 1 to 5, wherein L, D, L/D, n, D/d₁, and D/d₂ for said continuous multi-stage distillation column satisfy respectively 2500≦L≦5000, 210≦D≦800, 7≦L/D≦20, 40≦n≦90, 5≦D/d₁≦13, and 9≦D/d₂≦20,
7. the process according to any one of items 1 to 6, wherein the starting material aliphatic monohydric alcohol contains in a range of from 1.5 to 12% by weight of the dialkyl carbonate,
8. the process according to item 7, wherein the starting material aliphatic monohydric alcohol contains in a range of from 2 to 10% by weight of the dialkyl carbonate,
9. the process according to any one of items 1 to 8, wherein the starting material aliphatic monohydric alcohol is continuously introduced into said continuous multi-stage distillation-column from at least one inlet provided between the (2n/5)^{th} stage from the top of said continuous multi-stage distillation column and the (3n/5)^{th} stage from the top of said continuous multi-stage distillation column,
10. the process according to any one of items 1 to 9, wherein the tray is a sieve tray, said sieve tray having 100 to 1000 holes/m² in a sieve portion thereof,
11. the process according to any one of items 1 to 10, wherein the tray is a sieve tray, a cross-sectional area per hole of said sieve tray being in a range of from 0.5 to 5 cm².

### ADVANTAGEOUS EFFECT OF THE INVENTION

It has been discovered that by implementing the present invention, the dialkyl carbonate and the diol can be produced each with a high selectivity of not less than 95%, preferably not less than 97%, more preferably not less than 99%, on an industrial scale of not less than 2 ton 1 hr, preferably not less than 3 ton/hr, more preferably not less than 4 ton/hr, for the dialkyl carbonate, and not less than 1.3 ton/hr, preferably not less than 1.95 ton/hr, more preferably not less than 2.6 ton/hr, for the diol, stably for a prolonged period of time of not less than 1000 hours, preferably not less than 3000 hours, more preferably not less than 5000 hours, from a cyclic carbonate and an aliphatic monohydric alcohol.

### BRIEF DESCRIPTION OF DRAWING

FIG. 1 is an example of schematic drawing of a continuous multi-stage distillation column for carrying out the present invention, the distillation column having a number of stages n of trays as the internals (in FIG. 1, tray stages are shown schematically) provided inside a trunk portion thereof.

### DESCRIPTION OF REFERENCE NUMERALS

1: gas outlet, 2: liquid outlet, 3-a to 3-e: inlet, 4-a to 4-b: inlet, 5: end plate, 6: internal, 7: trunk portion, 10: continuous multi-stage distillation column, L: length (cm) of trunk portion, D: inside diameter (cm) trunk portion, d₁: inside diameter (cm) of gas outlet, d₂: inside diameter (cm) of liquid outlet.

### BEST MODE FOR CARRYING OUT THE INVENTION

In the following, the present invention is described in detail.

The reaction of the present invention is a reversible equilibrium transesterification reaction represented by the following formula in which a dialkyl carbonate (C) and a diol (D) are produced from a cyclic carbonate (A) and an aliphatic monohydric alcohol (B).

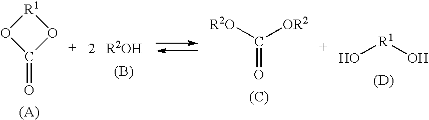

wherein R¹ represents a bivalent group —(CH₂)ₘ— (m is an integer from 2 to 6), one or more of the hydrogens thereof being optionally substituted with an alkyl group or aryl group having 1 to 10 carbon atoms. Moreover, R² represents a monovalent aliphatic group having 1 to 12 carbon atoms, one or more of the hydrogens thereof being optionally substituted with an alkyl group or aryl group having 1 to 10 carbon atoms.

The cyclic carbonate used as a starting material in the present invention is a compound represented by (A) in the above formula. For example, an alkylene carbonate such as ethylene carbonate or propylene carbonate, or 1,3-dioxacyclohexa-2-one, 1,3-dioxacyclohepta-2-one, or the like can be preferably used, ethylene carbonate or propylene carbonate being more preferably used due to ease of procurement and so on, and ethylene carbonate being particularly preferably used.

Moreover, the aliphatic monohydric alcohol used as the other starting material is a compound represented by (B) in the above formula. An aliphatic monohydric alcohol having a lower boiling point than that of the diol produced is used. Although possibly varying depending on the type of the cyclic carbonate used, examples are thus methanol, ethanol, propanol (isomers), allyl alcohol, butanol (isomers), 3-buten-1-ol, amyl alcohol (isomers), hexyl alcohol (isomers), heptyl alcohol (isomers), octyl alcohol (isomers), nonyl alcohol (isomers), decyl alcohol (isomers), undecyl alcohol (isomers), dodecyl alcohol (isomers), cyclopentanol, cyclohexanol, cycloheptanol, cyclooctanol, methylcyclopentanol (isomers), ethylcyclopentanol (isomers), methylcyclohexanol (isomers), ethylcyclohexanol (isomers), dimethylcyclohexanol (isomers), diethylcyclohexanol (isomers), phenylcyclohexanol (isomers), benzyl alcohol, phenethyl alcohol (isomers), phenylpropanol (isomers), and so on. Furthermore, these aliphatic monohydric alcohols may be substituted with substituents such as halogens, lower alkoxy groups, cyano groups, alkoxycarbonyl groups, aryloxycarbonyl groups, acyloxy groups, and nitro groups.

Of such aliphatic monohydric alcohols, ones preferably used are alcohols having 1 to 6 carbon atoms, more preferably alcohols having 1 to 4 carbon atoms, i.e. methanol, ethanol, propanol (isomers), and butanol (isomers). In the case of using ethylene carbonate or propylene carbonate as the cyclic carbonate, preferable aliphatic monohydric alcohols are methanol and ethanol, methanol being particularly preferable.

In the process of the present invention, a catalyst is made to be present in the reactive distillation column. The method of making the catalyst be present may be any method, but in the case, for example, of a homogeneous catalyst that dissolves in the reaction liquid under the reaction conditions, the catalyst can be made to be present in the liquid phase in the reactive distillation column by feeding the catalyst into the reactive distillation column continuously, or in the case of a heterogeneous catalyst that does not dissolve in the reaction liquid under the reaction conditions, the catalyst can be made to be present in the reaction system by disposing the catalyst as a solid in the reactive distillation column; these methods may also be used in combination.

In the case that a homogeneous catalyst is continuously fed into the reactive distillation column, the homogeneous catalyst may be fed in together with the cyclic carbonate and/or the aliphatic monohydric alcohol, or may be fed in at a different position to the starting materials. The reaction actually proceeds in the distillation column in a region below the position at which the catalyst is fed in, and hence it is preferable to feed the catalyst into a region between the top of the column and the position(s) at which the starting materials are fed in. The catalyst must be present in at least 5 stages, preferably at least 7 stages, more preferably at least 10 stages.

Moreover, in the case of using a heterogeneous solid catalyst, the catalyst must be present in at least 5 stages, preferably at least 7 stages, more preferably at least 10 stages. A solid catalyst that also has an effect as a packing in the distillation column may be used.

As the catalyst used in the present invention, any of various catalysts known from hitherto can be used. Examples of the catalysts include:

alkali metals and alkaline earth metals such as lithium, sodium, potassium, rubidium, cesium, magnesium, calcium, strontium, and barium;

basic compounds of alkali metals and alkaline earth metals such as hydrides, hydroxides, alkoxides, aryloxides, and amides;

basic compounds of alkali metals and alkaline earth metals such as carbonates, bicarbonates, and organic acid salts;

tertiary amines such as triethylamine, tributylamine, trihexylamine, and benzyldiethylamine;

nitrogen-containing heteroaromatic compounds such as N-alkylpyrroles, N-alkylindoles, oxazoles, N-alkylimidazoles, N-alkylpyrazoles, oxadiazoles, pyridine, alkylpyridines, quinoline, alkylquinolines, isoquinoline, alkylisoquinolines, acridine, alkylacridines, phenanthroline, alkylphenanthrolines, pyrimidine, alkylpyrimidines, pyrazine, alkylpyrazines, triazines, and alkyltriazines;

cyclic amidines such as diazobicycloundecene (DBU) and diazobicyclononene (DBN);

thallium compounds such as thallium oxide, thallium halides, thallium hydroxide, thallium carbonate, thallium nitrate, thallium sulfate, and thallium organic acid salts;

tin compounds such as tributylmethoxytin, tributylethoxytin, dibutyldimethoxytin, diethyldiethoxytin, dibutyldiethoxytin, dibutylphenoxytin, diphenylmethoxytin, dibutyltin acetate, tributyltin chloride, and tin 2-ethylhexanoate;

zinc compounds such as dimethoxyzinc, diethoxyzinc, ethylenedioxyzinc, and dibutoxyzinc;

aluminum compounds such as aluminum trimethoxide, aluminum triisopropoxide, and aluminum tributoxide;

titanium compounds such as tetramethoxytitanium, tetraethoxytitanium, tetrabutoxytitanium, dichlorodimethoxytitanium, tetraisopropoxytitanium, titanium acetate, and titanium acetylacetonate;

phosphorus compounds such as trimethylphosphine, triethylphosphine, tributylphosphine, triphenylphosphine, tributylmethylphosphonium halides, trioctylbutylphosphonium halides, and triphenylmethylphosphonium halides;

zirconium compounds such as zirconium halides, zirconium acetylacetonate, zirconium alkoxides, and zirconium acetate;

lead and lead-containing compounds, for example lead oxides such as PbO, PbO₂, and Pb₃O₄;

lead sulfides such as PbS, Pb₂S₃, and PbS2; lead hydroxides such as Pb(OH)₂, Pb₃O₂(OH)₂, Pb₂[PbO₂(OH)₂], and Pb₂O(OH)₂;

plumbites such as Na₂PbO₂, K₂PbO₂, NaHPbO₂, and KHPbO₂;

plumbates such as Na₂PbO₃, Na₂H₂PbO, K₂PbO₃, K₂[Pb(OH)₆], K₄PbO₄, Ca₂PbO₄, and CaPbO₃;

lead carbonates and basic salts thereof such as PbCO₃ and 2PbCO₃.Pb(OH)₂;

alkoxylead compounds and aryloxylead compounds such as Pb(OCH₃)₂, (CH₃O)Pb(OPh), and Pb(OPh)₂;

lead salts of organic acids, and carbonates and basic salts thereof, such as Pb(OCOCH₃)₂, Pb(OCOCH₃)₄, and Pb(OCOCH₃)₂.PbO.3H₂O;

organolead compounds such as Bu₄Pb, Ph₄Pb, Bu₃PbCl, Ph₃PbBr, Ph₃Pb (or Ph₆Pb₂), Bu₃PbOH, and Ph₂PbO (wherein Bu represents a butyl group, and Ph represents a phenyl group);

lead alloys such as Pb—Na, Pb—Ca, Pb—Ba, Pb—Sn, and Pb—Sb;

lead minerals such as galena and zinc blende; and

hydrates of such lead compounds.

In the case that the compound used dissolves in a starting material of the reaction, the reaction mixture, a reaction by-product or the like, the compound can be used as a homogeneous catalyst, whereas in the case that the compound does not dissolve, the compound can be used as a solid catalyst. Furthermore, it is also preferable to use, as a homogeneous catalyst, a mixture obtained by dissolving a compound as above in a starting material of the reaction, the reaction mixture, a reaction by-product or the like, or by reacting to bring about dissolution.

Furthermore, ion exchangers such as anion exchange resins having tertiary amino groups, ion exchange resins having amide groups, ion exchange resins having at least one type of exchange groups selected from sulfonate groups, carboxylate groups and phosphate groups, and solid strongly basic anion exchangers having quaternary ammonium groups as exchange groups; solid inorganic compounds such as silica, silica-alumina, silica-magnesia, aluminosilicates, gallium silicate, various zeolites, various metal-exchanged zeolites, and ammonium-exchanged zeolites, and so on can also be used as the catalyst.

As a solid catalyst, a particularly preferably used one is a solid strongly basic anion exchanger having quaternary ammonium groups as exchange groups, examples thereof including a strongly basic anion exchange resin having quaternary ammonium groups as exchange groups, a cellulose strongly basic anion exchanger having quaternary ammonium groups as exchange groups, and an inorganic carrier supported type strongly basic anion exchanger having quaternary ammonium groups as exchange groups. As a strongly basic anion exchange resin having quaternary ammonium groups as exchange groups, for example a styrene type strongly basic anion exchange resin or the like can be preferably used. A styrene type strongly basic anion exchange resin is a strongly basic anion exchange resin having a copolymer of styrene and divinylbenzene as a parent material, and having quaternary ammonium groups (type I or type II) as exchange groups, and can be schematically represented, for example, by the following formula;

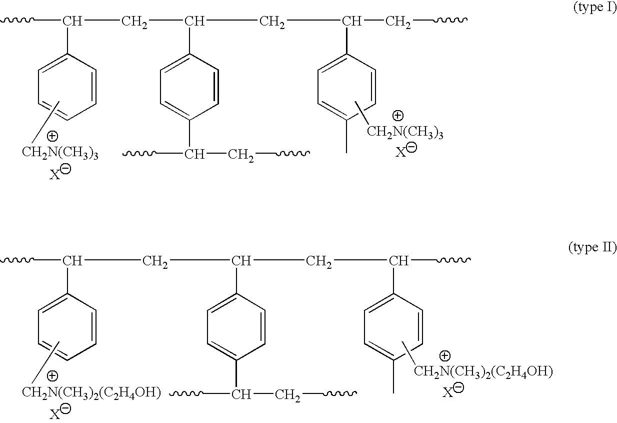

wherein X represents an anion; as X, generally at least one type of anion selected from F⁻, Cl⁻, Br⁻, I⁻, HCO₃⁻, CO₃²⁻, CH₃CO₂⁻, HCO₂⁻, IO₃⁻, BrO₃⁻, and ClO₃⁻ is used, preferably at least one type of anion selected from Cl⁻, Br⁻, HCO₃⁻, and CO₃²⁻. Moreover, as the structure of the resin parent material, either a gel type one or a macroreticular (MR) type one can be used, the MR type being particularly preferable due to the organic solvent resistance being high.

An example of a cellulose strongly basic anion exchanger having quaternary ammonium groups as exchange groups is cellulose having —OCH₂CH₂NR₃X exchange groups obtained by converting some or all of the —OH groups in the cellulose into trialkylaminoethyl groups. Here, R represents an alkyl group; methyl, ethyl, propyl, butyl or the like is generally used, preferably methyl or ethyl. Moreover, X represents an anion as above.

An inorganic carrier supported type strongly basic anion exchanger having quaternary ammonium groups as exchange groups that can be used in the present invention means an inorganic carrier that has had —O(CH₂)ₙNR₃X quaternary ammonium groups introduced thereto by modifying some or all of the —OH surface hydroxyl groups of the inorganic carrier. Here, R and X are defined above. n is generally an integer from 1 to 6, preferably n=2. Examples of the inorganic carrier include silica, alumina, silica-alumina, titania, a zeolite, or the like, it being preferable to use silica, alumina, or silica-alumina, particularly preferably silica. Any method can be used as the method of modifying the surface hydroxyl groups of the inorganic carrier.

As the solid strongly basic anion exchanger having quaternary ammonium groups as exchange groups, a commercially available one may be used. In this case, the anion exchanger may also be used as the transesterification catalyst after being subjected to ion exchange with a desired anionic species in advance as pretreatment.

Moreover, a solid catalyst containing a macroreticular or gel-type organic polymer having bonded thereto heterocyclic groups each containing at least one nitrogen atom, or an inorganic carrier having bonded thereto heterocyclic groups each containing at least one nitrogen atom can also be preferably used as the transesterification catalyst. Furthermore, a solid catalyst in which some or all of these nitrogen-containing heterocyclic groups have been converted into a quaternary salt can be similarly used. Note that a solid catalyst such as an ion exchanger may also act as a packing in the present invention.

The amount of the catalyst used in the present invention varies depending on the type of the catalyst used, but in the case of continuously feeding in a homogeneous catalyst that dissolves in the reaction liquid under the reaction conditions, the amount used is generally in a range of from 0.0001 to 50% by weight, preferably from 0.005 to 20% by weight, more preferably from 0.01 to 10% by weight, as a proportion of the total weight of the cyclic carbonate and the aliphatic monohydric alcohol fed in as the starting materials. Moreover, in the case of using a solid catalyst installed in the distillation column, the catalyst is preferably used in an amount in a range of from 0.01 to 75 vol %, more preferably from 0.05 to 60 vol %, yet more preferably from 0.1 to 60 vol %, based on the empty column volume of the distillation column.

When continuously feeding the cyclic carbonate into the continuous multi-stage distillation column constituting the reactive distillation column in the present invention, it is important for the cyclic carbonate to be fed into a specified stage. Specifically, the starting material cyclic carbonate must be continuously introduced into the continuous multi-stage distillation column from at least one inlet provided between the 3^{rd} stage from the top of the continuous multi-stage distillation column and the (n/3)^{th} stage from the top of the continuous multi-stage distillation column. This is important to ensure that high boiling point compounds such as the cyclic carbonate and the diol are not contained in a column top component in stages above the cyclic carbonate inlet(s). For this reason, there are preferably not less than 3 stages, more preferably 4 to 10 stages, yet more preferably 5 to 8 stages, above the cyclic carbonate inlet(s).

A cyclic carbonate preferably used in the present invention is one not containing a halogen that has been produced through reaction between, for example, an alkylene oxide such as ethylene oxide, propylene oxide or styrene oxide and carbon dioxide. A cyclic carbonate containing small amounts of such raw material compounds, the diol, and so on may thus be used as a starting material in the present invention.

In the present invention, the starting material aliphatic monohydric alcohol must contain in a range of from 1 to 15% by weight of the dialkyl carbonate based on the total weight of the aliphatic monohydric alcohol and the dialkyl carbonate. It is more preferable to use the aliphatic monohydric alcohol having this dialkyl carbonate content in a range of from 1.5 to 12% by weight, yet more preferably from 2 to 10% by weight.

When carrying out the present reaction industrially, besides fresh cyclic carbonate and/or aliphatic monohydric alcohol newly introduced into the reaction system, matter having the cyclic carbonate and/or the aliphatic monohydric alcohol as a main component thereof recovered from this process and/or another process can also be preferably used for the starting materials. It is an excellent characteristic feature of the present invention that this is possible. An example of another process is a process in which the diaryl carbonate is produced from the dialkyl carbonate and an aromatic monohydroxy compound, the aliphatic monohydric alcohol being by-produced in this process and recovered. The recovered by-produced aliphatic monohydric alcohol generally contains the dialkyl carbonate, but it has been discovered that in the case that the content of the dialkyl carbonate is in a range as above, excellent effects of the present invention can be achieved. The recovered by-produced aliphatic monohydric alcohol may further contain the aromatic monohydroxy compound, an alkyl aryl ether, small amounts of an alkyl aryl carbonate and the diaryl carbonate, and so on. The by-produced aliphatic monohydric alcohol may be used as is as the starting material in the present invention, or may be used as the starting material after the amount of contained matter having a higher boiling point than the aliphatic monohydric alcohol has been reduced through distillation or the like.

When continuously feeding the aliphatic monohydric alcohol into the continuous multi-stage distillation column constituting the reactive distillation column in the present invention, it is important for the aliphatic monohydric alcohol to be fed into a specified stage. Specifically, in the present invention, the starting material aliphatic monohydric alcohol must be continuously introduced into the continuous multi-stage distillation column from at least one inlet provided between the (n/3)^{th} stage from the top of the continuous multi-stage distillation column and the (2n/3)^{th} stage from the top of the continuous multi-stage distillation column. It has been discovered that, because the aliphatic monohydric alcohol used as the starting material in the present invention contains a specified amount of the dialkyl carbonate, excellent effects of the present invention can be achieved by making the aliphatic monohydric alcohol inlet(s) be in a stage within this specified range. More preferably, the aliphatic monohydric alcohol is continuously introduced into the continuous multi-stage distillation column from at least one inlet provided between the (2n/5)^{th} stage from the top of the continuous multi-stage distillation column and the (3n/5)^{th} stage from the top of the continuous multi-stage distillation column.

The starting materials are fed continuously into the distillation column in a liquid form, in a gaseous form, or as a mixture of a liquid and a gas. Other than feeding the starting materials into the distillation column in this way, it is also preferable to additionally feed in a gaseous starting material intermittently or continuously from a central portion and/or a lower portion of the distillation column. Moreover, another preferable method is one in which the cyclic carbonate is continuously fed in a liquid form or a gas/liquid mixed form into a stage of the distillation column above the stages in which the catalyst is present, and the aliphatic monohydric alcohol is continuously fed into the distillation column in a gaseous form and/or a liquid form from at least one inlet provided in a stage of the distillation column as described above. The starting materials are preferably made to contact the catalyst in a region of at least 5 stages, preferably at least 7 stages, more preferably at least 10 stages, of the distillation column.

In the present invention, a ratio between the amounts of the cyclic carbonate and the aliphatic monohydric alcohol fed into the reactive distillation column varies according to the type and amount of the transesterification catalyst and the reaction conditions, but a molar ratio of the aliphatic monohydric alcohol to the cyclic carbonate fed in is generally in a range of from 0.01 to 1000 times. To increase the cyclic carbonate conversion, it is preferable to feed in the aliphatic monohydric alcohol in an excess of at least 2 times the number of mols of the cyclic carbonate, but if an amount of the aliphatic monohydric alcohol used is too great, then it is necessary to make the apparatus larger. For such reasons, the molar ratio of the aliphatic monohydric alcohol to the cyclic carbonate is preferably in a range of from 2 to 20, more preferably from 3 to 15, yet more preferably from 5 to 12. Furthermore, if much unreacted cyclic carbonate remains, then the unreacted cyclic carbonate may react with the product diol to by-produce oligomers such as a dimer or a trimer, and hence in industrial implementation, it is preferable to reduce the amount of unreacted cyclic carbonate remaining as much as possible. In the process of the present invention, even if the above molar ratio is not more than 10, the cyclic carbonate conversion can be made to be not less than 98%, preferably not less than 99%, more preferably not less than 99.9%. This is another characteristic feature of the present invention.

In the present invention, preferably not less than 2 ton/hr of the dialkyl carbonate is continuously produced; the minimum amount of the cyclic carbonate continuously fed in to achieve this is generally 2.2 P ton/hr, preferably 2.1 P ton/hr, more preferably 2.0 P ton/hr, relative to the amount P (ton/hr) of the dialkyl carbonate to be produced. In a yet more preferable case, this amount can be made to be less than 1.9 P ton/hr.

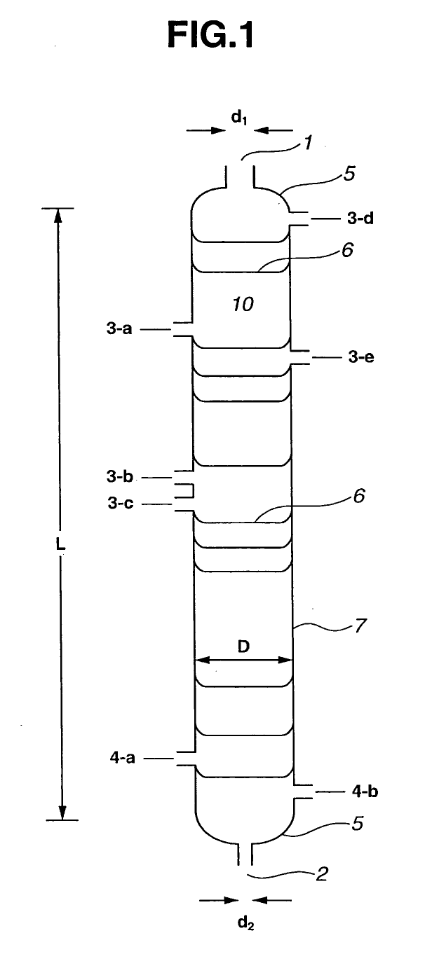

FIG. 1 illustrates an example of schematic drawing of the continuous multi-stage distillation column for carrying out the production process according to the present invention. Here, the continuous multi-stage distillation column 10 used in the present invention comprises a tray column type distillation column which has a structure having a pair of end plates 5 above and below a cylindrical trunk portion 7 having a length L (cm) and an inside diameter D (cm), and having thereinside an internal with a number of stages n, said internal being a tray having a plurality of holes, and which further has a gas outlet 1 having an inside diameter d₁ (cm) at a top of the column or in an upper portion of the column near to the top, a liquid outlet 2 having an inside diameter d₂ (cm) at a bottom of the column or in a lower portion of the column near to the bottom, at least one first inlet 3 (a, e) provided below the gas outlet 1 between the 3^{rd} stage from the top of the continuous multi-stage distillation column and the (n/3)^{th} stage from the top of the continuous multi-stage distillation column, and at least one second inlet 3(b, c) and 4 (a, b) provided above the liquid outlet 2 between the (n/3)^{th} stage from the top of the continuous multi-stage distillation column and the (2n/3)^{th} stage from the top of the continuous multi-stage distillation column, and moreover must be made to satisfy various conditions so as to be able to carry out not only distillation but also reaction at the same time so as to be able to produce preferably not less than 2 ton/hr of the dialkyl carbonate and/or preferably not less than 1.3 ton/hr of the diol stably for a prolonged period of time. Note that FIG. 1 illustrates merely one embodiment of the continuous multi-stage distillation column according to the present invention, and hence the arrangement of the tray stages is not limited to that shown in FIG. 1.

The continuous multi-stage distillation column according to the present invention satisfies not only conditions from the perspective of the distillation function, but rather these conditions are combined with conditions required so as make the reaction proceed stably with a high conversion and high selectivity, specifically:

(1) the length L (cm) must satisfy the formula (1);

2100≦L≦8000 (1)

(2) the inside diameter D (cm) of the column must satisfy the formula (2);

180≦D≦2000 (2),

(3) a ratio of the length L (cm) to the inside diameter D (cm) of the column must satisfy the formula (3);

4≦L/D≦40 (3),

(4) the number of stages n must satisfy the formula (4);

10≦n≦120 (4),

(5) a ratio of the inside diameter D (cm) of the column to the inside diameter d₁ (cm) of the gas outlet must satisfy the formula (5);

3≦D/d₁≦20 (5), and

(6) a ratio of the inside diameter D (cm) of the column to the inside diameter d₂ (cm) of the liquid outlet must satisfy the formula (6);

5≦D/d₂≦30 (6).

Note that the term “the top of the column or the upper portion of the column near to the top” used in the present invention means the portion from the top of the column downward as far as approximately 0.25 L, and the term “the bottom of the column or the lower portion of the column near to the bottom” means the portion from the bottom of the column upward as far as approximately 0.25 L. Here, “L” is defined as above.

It has been discovered that by using the continuous multi-stage distillation column that simultaneously satisfies the formulae (1), (2), (3), (4), (5) and (6), and in which the starting material cyclic carbonate and aliphatic monohydric alcohol inlets are respectively within the specified ranges described above, and moreover using an aliphatic monohydric alcohol containing a specified amount in a range of from 1 to 15% by weight of the dialkyl carbonate as the starting material, the dialkyl carbonate and the diol can be produced on an industrial scale of preferably not less than 2 ton/hr of the dialkyl carbonate and/or preferably not less than 1.3 ton/hr of the diol with a high conversion, high selectivity, and high productivity stably for a prolonged period of time of, for example, not less than 1000 hours, preferably not less than 3000 hours, more preferably not less than 5000 hours, from the cyclic carbonate and the aliphatic monohydric alcohol. The reason why it has become possible to produce the dialkyl carbonate and the diol on an industrial scale with such excellent effects by implementing the process of the present invention is not clear, but this is supposed to be due to a composite effect brought about when the above conditions are combined. Preferable ranges for the respective factors are described below.

If L (cm) is less than 2100, then the conversion decreases and hence it is not possible to attain the desired production amount. Moreover, to keep down the equipment cost while securing the conversion enabling the desired production amount to be attained, L must be made to be not more than 8000. A more preferable range for L (cm) is 2300≦L≦6000, with 2500≦L≦5000 being yet more preferable.

If D (cm) is less than 180, then it is not possible to attain the desired production amount. Moreover, to keep down the equipment cost while attaining the desired production amount, D must be made to be not more than 2000. A more preferable range for D (cm) is 200≦D≦1000, with 210≦D≦800 being yet more preferable.

If L/D is less than 4 or greater than 40, then stable operation becomes difficult. In particular, if L/D is greater than 40, then the pressure difference between the top and bottom of the column becomes too great, and hence prolonged stable operation becomes difficult. Moreover, it becomes necessary to increase the temperature in the lower portion of the column, and hence side reactions become liable to occur, bringing about a decrease in the selectivity. A more preferable range for L/D is 5≦L/D≦30, with 7≦L/D≦20 being yet more preferable.

If n is less than 10, then the conversion decreases and hence it is not possible to attain the desired production amount. Moreover, to keep down the equipment cost while securing the conversion enabling the desired production amount to be attained, n must be made to be not more than 120. Furthermore, if n is greater than 120, then the pressure difference between the top and bottom of the column becomes too great, and hence prolonged stable operation becomes difficult. Moreover, it becomes necessary to increase the temperature in the lower portion of the column, and hence side reactions become liable to occur, bringing about a decrease in the selectivity. A more preferable range for n is 30≦n≦100, with 40≦n≦90 being yet more preferable.

If D/d₁ is less than 3, then the equipment cost becomes high. Moreover, a large amount of a gaseous component is readily released to the outside of the system, and hence stable operation becomes difficult. If D/d₁ is greater than 20, then the gaseous component withdrawal amount becomes relatively low, and hence stable operation becomes difficult, and moreover a decrease in the conversion is brought about. A more preferable range for D/d₁ is 4≦D/d₁≦15, with 5≦D/d₁≦13 being yet more preferable.

If D/d₂ is less than 5, then the equipment cost becomes high. Moreover, the liquid withdrawal amount becomes relatively high, and hence stable operation becomes difficult. If D/d₂ is greater than 30, then the flow rate through the liquid outlet and piping becomes excessively fast, and hence erosion becomes liable to occur, bringing about corrosion of the apparatus. A more preferable range for D/d₂ is 7≦D/d₂≦25, with 9≦D/d₂≦20 being yet more preferable.

Furthermore, it has been found that in the present invention it is further preferable for d₁ and d₂ to satisfy the formula (7);

1≦d₁/d₂≦5 (7).

The term “prolonged stable operation” used in the present invention means that the continuous multi-stage distillation column can be operated continuously in a steady state based on the operating conditions with no flooding or weeping, clogging of piping, or erosion for not less than 1000 hours, preferably not less than 3000 hours, more preferably not less than 5000 hours, and predetermined amounts of the dialkyl carbonate and the diol can be produced while maintaining a high conversion, high selectivity, and high productivity.

A characteristic feature of the present invention is that the dialkyl carbonate and the diol can be produced stably for a prolonged period of time each with high selectivity and preferably with high productivity for the dialkyl carbonate of not less than 2 ton/hr and high productivity for the diol of not less than 1.3 ton/hr. The dialkyl carbonate and the diol are more preferably produced in an amount of not less than 3 ton/hr and not less than 1.95 ton/hr respectively, yet more preferably not less than 4 ton/hr and not less than 2.6 ton/hr respectively. Moreover, another characteristic feature of the present invention is that in the case that L, D, L/D, n, D/d₁, and D/d₂ for the continuous multi-stage distillation column satisfy respectively 2300≦L≦6000, 200≦D≦1000, 5≦L/D≦30, 30≦n≦100, 4≦D/d₁≦15, and 7≦D/d₂≦25, not less than 2.5 ton/hr, preferably not less than 3 ton/hr, more preferably not less than 3.5 ton/hr of the dialkyl carbonate, and not less than 1.6 ton/hr, preferably not less than 1.95 ton/hr, more preferably not less than 2.2 ton/hr of the diol can be produced. Furthermore, another characteristic feature of the present invention is that in the case that L, D, L/D, n, D/d₁, and D/d₂ for the continuous multi-stage distillation column satisfy respectively 2500≦L≦5000, 210≦D≦800, 7≦L/D≦20, 40≦n≦90, 5≦D/d₁≦13, and 9≦D/d₂≦20, not less than 3 ton/hr, preferably not less than 3.5 ton/hr, more preferably not less than 4 ton/hr of the dialkyl carbonate, and not less than 1.95 ton/hr, preferably not less than 2.2 ton/hr, more preferably not less than 2.6 ton/hr of the diol can be produced.

The “selectivity” for each of the dialkyl carbonate and the diol in the present invention is based on the cyclic carbonate reacted. In the present invention, a high selectivity of not less than 95% can generally be attained, preferably not less than 97%, more preferably not less than 99%. Moreover, the “conversion” in the present invention generally indicates the cyclic carbonate conversion, in the present invention it being possible to make the cyclic carbonate conversion be not less than 95%, preferably not less than 97%, more preferably not less than 99%, yet more preferably not less than 99.5%, still more preferably not less than 99.9%. It is one of the excellent characteristic features of the present invention that a high conversion can be maintained while maintaining high selectivity in this way.

The continuous multi-stage distillation column used in the present invention comprises a tray column type distillation column including trays having a plurality of holes trays as the internal. The term “internal” used in the present invention means the parts in the distillation column where gas and liquid are actually brought into contact with one another. Examples of the trays include a bubble-cap tray, a sieve tray, a ripple tray, a ballast tray, a valve tray, a counterflow tray, a Unifrax tray, a Superfrac tray, a Maxfrac tray, a dual flow tray, a grid plate tray, a turbogrid plate tray, a Kittel tray, or the like. Moreover, in the present invention, the multi-stage distillation column having both a tray portion and a portion packed with packings in part of the tray stage portion can also be used. Examples of the packings include a random packing such as a Raschig ring, a Lessing ring, a Pall ring, a Berl saddle, a Intalox saddle, a Dixon packing, a McMahon packing or Heli-Pak, or a structured packing such as Mellapak, Gempak, Techno-pack, Flexipac, a Sulzer packing, a Goodroll packing or Glitschgrid. Furthermore, the term “number of stages n of the internal” used in the present invention means the number of trays in the case of trays, and the theoretical number of stages in the case of packings.

The number of stages n in the case of a multi-stage distillation column having both a tray portion and a portion packed with the packing is thus the sum of the number of trays and the theoretical number of stages.

For the reaction between the cyclic carbonate and the aliphatic monohydric alcohol in the present invention, it has been discovered that a high conversion, high selectivity, and high productivity can be attained even if a plate type continuous multi-stage distillation column in which the internals comprise the trays having a predetermined number of stages or the packings as a part of internals is used, but the plate type distillation column in which the internals are trays is preferable. Furthermore, it has been discovered that a sieve tray having a sieve portion and a downcomer portion is particularly good as the tray in terms of the relationship between performance and equipment cost. It has also been discovered that the sieve tray preferably has 100 to 1000 holes/m² in the sieve portion. A more preferable number of holes is 120 to 900 holes/m², yet more preferably 150 to 800 holes/m². Moreover, it has been discovered that a cross-sectional area per hole of the sieve tray is preferably in a range of from 0.5 to 5 cm². A more preferable cross-sectional area per hole is 0.7 to 4 cm², yet more preferably 0.9 to 3 cm². Furthermore, it has been discovered that it is particularly preferable if the sieve tray has 100 to 1000 holes/m² in the sieve portion, and the cross-sectional area per hole is in a range of from 0.5 to 5 cm². The number of holes in the sieve portion may be the same for all of the sieve trays, or may differ. In the case that the number of holes or the cross-sectional area per hole differs between the sieve trays, the total hole area of the holes present in one tray stage may differ between the stages; in this case, S (m²) in the present invention means the average value of the total hole area for the stages. It has been shown that by adding the above conditions to the continuous multi-stage distillation column, the object of the present invention can be attained more easily.

When carrying out the present invention, the dialkyl carbonate and the diol are continuously produced by continuously feeding the cyclic carbonate and the aliphatic monohydric alcohol as the starting materials into the continuous multi-stage distillation column in which the catalyst is present, carrying out reaction and distillation simultaneously in the column, continuously withdrawing a low boiling point reaction mixture containing the produced dialkyl carbonate from an upper portion of the column in a gaseous form, and continuously withdrawing a high boiling point reaction mixture containing the diol from a lower portion of the column in a liquid form.

The reaction time for the transesterification reaction carried out in the present invention is considered to equate to the average residence time of the reaction liquid in the continuous multi-stage distillation column. The reaction time varies depending on the form of the internals in the distillation column and the number of stages, the amounts of the starting materials fed in, the type and amount of the catalyst, the reaction conditions, and so on. The reaction time is generally in a range of from 0.1 to 20 hours, preferably from 0.5 to 15 hours, more preferably from 1 to 10 hours.

The reaction temperature varies depending on the type of the starting material compounds used, and the type and amount of the catalyst. The reaction temperature is generally in a range of from 30 to 300° C. It is preferable to increase the reaction temperature so as to increase the reaction rate. However, if the reaction temperature is too high, then side reactions become liable to occur. The reaction temperature is thus preferably in a range of from 40 to 250° C., more preferably from 50 to 200° C., yet more preferably from 60 to 150° C. In the present invention, the reactive distillation can be carried out with the column bottom temperature set to not more than 150° C., preferably not more than 130° C., more preferably not more than 110° C., yet more preferably not more than 100° C. An excellent characteristic feature of the present invention is that a high conversion, high selectivity, and high productivity can be attained even with such a low column bottom temperature. Moreover, the reaction pressure varies depending on the type of the starting material compounds used and the composition therebetween, the reaction temperature, and so on. The reaction pressure may be any of a reduced pressure, normal pressure, or an applied pressure, and is generally in a range of from 1 to 2×10⁷ Pa, preferably from 10³ to 10⁷ Pa, more preferably from 10⁴ to 5×10⁶ Pa.

The material constituting the continuous multi-stage distillation column used in the present invention is generally a metallic material such as carbon steel or stainless steel. In terms of the quality of the dialkyl carbonate and the diol to be produced, stainless steel is preferable.

### EXAMPLES

Following is a more detailed description of the present invention through examples. However, the present invention is not limited to the following examples.

### Example 1

### Continuous Multi-Stage Distillation Column

A continuous multi-stage distillation column as shown in FIG. 1 having L=3300 cm, D=300 cm, L/D=11, n=60, D/d₁=7.5, and D/d₂=12 was used. In this example, as the internal, sieve trays each having a cross-sectional area per hole in the sieve portion thereof of approximately 1.3 cm² and a number of holes of approximately 180 to 320/m² were used.

### <Reactive Distillation>

3.27 Ton/hr of ethylene carbonate in a liquid form was continuously introduced into the distillation column from an inlet (3-a) provided at the 5^{th} stage from the top. 3.238 Ton/hr of methanol in a gaseous form (containing 8.96% by weight of dimethyl carbonate) and 7.489 ton/hr of methanol in a liquid form (containing 6.66% by weight of dimethyl carbonate) were respectively continuously introduced into the distillation column from inlets (3-b and 3-c) provided at the 30^{th} stage from the top. The content of dimethyl carbonate in the starting material methanol was 7.35% by weight. Further, a molar ratio of the starting materials introduced into the distillation column was methanol/ethylene carbonate=8.36.

The catalyst used was obtained by adding 4.8 ton of ethylene glycol to 2.5 ton of KOH (48% by weight aqueous solution), heating to approximately 130° C., gradually reducing the pressure, and carrying out heat treatment for approximately 3 hours at approximately 1300 Pa, so as to produce a homogeneous solution. This catalyst solution was continuously introduced into the distillation column from an inlet (3-e) provided at the 6^{th} stage from the top (K concentration: 0.1% by weight based on ethylene carbonate fed in). Reactive distillation was carried out continuously under conditions of a column bottom temperature of 98° C., a column top pressure of approximately 1.118×10⁵ Pa, and a reflux ratio of 0.42.

It was possible to attain stable steady state operation after 24 hours. A low boiling point reaction mixture withdrawn from the top 1 of the column in a gaseous form was cooled using a heat exchanger and thus turned into a liquid. The liquid low boiling point reaction mixture, which was continuously withdrawn from the distillation column at 10.678 ton/hr, contained 4.129 ton/hr of dimethyl carbonate, and 6.549 ton/hr of methanol. A liquid continuously withdrawn from the bottom 2 of the column at 3.382 ton/hr contained 2.356 ton/hr of ethylene glycol, 1.014 ton/hr of methanol, and 4 kg/hr of unreacted ethylene carbonate. Excluding the dimethyl carbonate contained in the starting material, the actual produced amount of dimethyl carbonate was 3.340 ton/hr, and excluding the ethylene glycol contained in the catalyst solution, the actual produced amount of ethylene glycol was 2.301 ton/hr. The ethylene carbonate conversion was 99.88%, the dimethyl carbonate selectivity was not less than 99.99%, and the ethylene glycol selectivity was not less than 99.99%.

Prolonged continuous operation was carried out under these conditions. After 500 hours, 2000 hours, 4000 hours, 5000 hours, and 6000 hours, the actual produced amounts per hour were 3.340 ton, 3.340 ton, 3.340 ton, 3.340 ton, and 3.340 ton respectively for dimethyl carbonate, and 2.301 ton, 2.301 ton, 2.301 ton, 2.301 ton, and 2.301 ton respectively for ethylene glycol, the ethylene carbonate conversions were respectively 99.90%, 99.89%, 99.89%, 99.88%, and 99.88%, the dimethyl carbonate selectivities were respectively not less than 99.99%, not less than 99.99%, not less than 99.99%, not less than 99.99%, and not less than 99.99%, and the ethylene glycol selectivities were respectively not less than 99.99%, not less than 99.99%, not less than 99.99%, not less than 99.99%, and not less than 99.99%.

### Example 2

Reactive distillation was carried out under the following conditions using the same continuous multi-stage distillation column as in Example 1. 2.61 Ton/hr of ethylene carbonate in a liquid form was continuously introduced into the distillation column from the inlet (3-a) provided at the 5^{th} stage from the top. 4.233 Ton/hr of methanol in a gaseous form (containing 2.41% by weight of dimethyl carbonate) and 4.227 ton/hr of methanol in a liquid form (containing 1.46% by weight of dimethyl carbonate) were respectively continuously introduced into the distillation column from the inlets (3-b and 3-c) provided at the 30^{th} stage from the top. The content of dimethyl carbonate in the starting material methanol was 2.02% by weight. Further, a molar ratio of the starting materials introduced into the distillation column was methanol/ethylene carbonate=8.73. The catalyst was made to be the same as in Example 1, and was continuously fed into the distillation column. Reactive distillation was carried out continuously under conditions of a column bottom temperature of 93° C., a column top pressure of approximately 1.046×10⁵ Pa, and a reflux ratio of 0.48.

It was possible to attain stable steady state operation after 24 hours. A low boiling point reaction mixture withdrawn from the top 1 of the column in a gaseous form was cooled using a heat exchanger and thus turned into a liquid. The liquid low boiling point reaction mixture, which was continuously withdrawn from the distillation column at 8.17 ton/hr, contained 2.84 ton/hr of dimethyl carbonate, and 5.33 ton/hr of methanol. A liquid continuously withdrawn from the bottom 2 of the column at 2.937 ton/hr contained 1.865 ton/hr of ethylene glycol, 1.062 ton/hr of methanol, and 0.2 kg/hr of unreacted ethylene carbonate. Excluding the dimethyl carbonate contained in the starting material, the actual produced amount of dimethyl carbonate was 2.669 ton/hr, and excluding the ethylene glycol contained in the catalyst solution, the actual produced amount of ethylene glycol was 1.839 ton/hr. The ethylene carbonate conversion was 99.99%, the dimethyl carbonate selectivity was not less than 99.99%, and the ethylene glycol selectivity was not less than 99.99%.

Prolonged continuous operation was carried out under these conditions. After 1000 hours, 2000 hours, 3000 hours, and 5000 hours, the actual produced amounts per hour were 2.669 ton, 2.669 ton, 2.669 ton, and 2.669 ton respectively for dimethyl carbonate, and 1.839 ton, 1.839 ton, 1.839 ton, and 1.839 ton respectively for ethylene glycol, the ethylene carbonate conversions were respectively 99.99%, 99.99%, 99.99%, and 99.99%, the dimethyl carbonate selectivities were respectively not less than 99.99%, not less than 99.99%, not less than 99.99%, and not less than 99.99%, and the ethylene glycol selectivities were respectively not less than 99.99%, not less than 99.99%, not less than 99.99%, and not less than 99.99%.

### Example 3

A continuous multi-stage distillation column as shown in FIG. 1 having L=3300 cm, D=300 cm, L/D=11, n=60, D/d₁=7.5, and D/d₂=12 was used. In this example, as the internal, sieve trays each having a cross-sectional area per hole in the sieve portion thereof of approximately 1.3 cm² and a number of holes of approximately 220 to 340/m² were used.

3.773 Ton/hr of ethylene carbonate in a liquid form was continuously introduced into the distillation column from the inlet (3-a) provided at the 5^{th} stage from the top. 3.736 Ton/hr of methanol in a gaseous form (containing 8.97% by weight of dimethyl carbonate) and 8.641 ton/hr of methanol in a liquid form (containing 6.65% by weight of dimethyl carbonate) were respectively continuously introduced into the distillation column from the inlets (3-b and 3-c) provided at the 30^{th} stage from the top. The content of dimethyl carbonate in the starting material methanol was 7.35% by weight. Further, a molar ratio of the starting materials introduced into the distillation column was methanol/ethylene carbonate=8.73. The catalyst was made to be the same as in Example 1, and was continuously fed into the distillation column. Reactive distillation was carried out continuously under conditions of a column bottom temperature of 98° C., a column top pressure of approximately 1.118×10⁵ Pa, and a reflux ratio of 0.42.

It was possible to attain stable steady state operation after 24 hours. A low boiling point reaction mixture withdrawn from the top of the column in a gaseous form was cooled using a heat exchanger and thus turned into a liquid. The liquid low boiling point reaction mixture, which was continuously withdrawn from the distillation column at 12.32 ton/hr, contained 4.764 ton/hr of dimethyl carbonate, and 7.556 ton/hr of methanol. A liquid continuously withdrawn from the bottom of the column at 3.902 ton/hr contained 2.718 ton/hr of ethylene glycol, 1.17 ton/hr of methanol, and 4.6 kg/hr of unreacted ethylene carbonate. Excluding the dimethyl carbonate contained in the starting material, the actual produced amount of dimethyl carbonate was 3.854 ton/hr, and excluding the ethylene glycol contained in the catalyst solution, the actual produced amount of ethylene glycol was 2.655 ton/hr. The ethylene carbonate conversion was 99.88%, the dimethyl carbonate selectivity was not less than 99.99%, and the ethylene glycol selectivity was not less than 99.99%.

Prolonged continuous operation was carried out under these conditions. After 1000 hours, 2000 hours, 3000 hours, and 5000 hours, the actual produced amounts per hour were 3.854 ton, 3.854 ton, 3.854 ton, and 3.854 ton respectively for dimethyl carbonate, and 2.655 ton, 2.655 ton, 2.655 ton, and 2.655 ton respectively for ethylene glycol, the ethylene carbonate conversions were respectively 99.99%, 99.99%, 99.99%, and 99.99%, the dimethyl carbonate selectivities were respectively not less than 99.99%, not less than 99.99%, not less than 99.99%, and not less than 99.99%, and the ethylene glycol selectivities were respectively not less than 99.99%, not less than 99.99%, not less than 99.99%, and not less than 99.99%.

### Example 4

A continuous multi-stage distillation column as shown in FIG. 1 having L=3300 cm, D=300 cm, L/D=11, n=60, D/d₁=7.5, and D/d₂=12 was used. In this example, as the internal, sieve trays each having a cross-sectional area per hole in the sieve portion thereof of approximately 1.3 cm² and a number of holes of approximately 240 to 360/m² were used.

7.546 Ton/hr of ethylene carbonate in a liquid form was continuously introduced into the distillation column from the inlet (3-a) provided at the 5^{th} stage from the top. 7.742 Ton/hr of methanol in a gaseous form (containing 8.95% by weight of dimethyl carbonate) and 17.282 ton/hr of methanol in a liquid form (containing 6.66% by weight of dimethyl carbonate) were respectively continuously introduced into the distillation column from the inlets (3-b and 3-c) provided at the 30^{th} stage from the top. The content of dimethyl carbonate in the starting material methanol was 7.35% by weight. Further, a molar ratio of the starting materials introduced into the distillation column was methanol/ethylene carbonate=8.36. The catalyst was made to be the same as in Example 1, and was continuously fed into the distillation column. Reactive distillation was carried out continuously under conditions of a column top temperature of 65° C., a column top pressure of approximately 1.118×10⁵ Pa, and a reflux ratio of 0.42.

It was possible to attain stable steady state operation after 24 hours. A low boiling point reaction mixture withdrawn from the top 1 of the column in a gaseous form was cooled using a heat exchanger and thus turned into a liquid. The liquid low boiling point reaction mixture, which was continuously withdrawn from the distillation column at 24.641 ton/hr, contained 9.527 ton/hr of dimethyl carbonate, and 15.114 ton/hr of methanol. A liquid continuously withdrawn from the bottom 2 of the column at 7.804 ton/hr contained 5.436 ton/hr of ethylene glycol, 2.34 ton/hr of methanol, and 23 kg/hr of unreacted ethylene carbonate. Excluding the dimethyl carbonate contained in the starting material, the actual produced amount of dimethyl carbonate was 7.708 ton/hr, and excluding the ethylene glycol contained in the catalyst solution, the actual produced amount of ethylene glycol was 5.31 ton/hr. The ethylene carbonate conversion was 99.7%, the dimethyl carbonate selectivity was not less than 99.99%, and the ethylene glycol selectivity was not less than 99.99%.

Prolonged continuous operation was carried out under these conditions. After 1000 hours, the actual produced amount per hour was 7.708 ton for dimethyl carbonate, and 5.31 ton for ethylene glycol, the ethylene carbonate conversion was 99.8%, the dimethyl carbonate selectivity was not less than 99.99%, and the ethylene glycol selectivity was not less than 99.99%.

### INDUSTRIAL APPLICABILITY

According to the present invention, it has been discovered that the dialkyl carbonate and the diol can be produced each with a high selectivity of not less than 98%, preferably not less than 99%, more preferably not less than 99.9%, on an industrial scale of not less than 2 ton/hr, preferably not less than 3 ton/hr, more preferably not less than 4 ton/hr, for the dialkyl carbonate, and not less than 1.3 ton/hr, preferably not less than 1.95 ton/hr, more preferably not less than 2.6 ton/hr, for the diol, stably for a prolonged period of time of not less than 1000 hours, preferably not less than 3000 hours, more preferably not less than 5000 hours, from the cyclic carbonate and the aliphatic monohydric alcohol.

1. An industrial process for the production of a dialkyl carbonate and a diol in which the dialkyl carbonate and the diol are continuously produced through a reactive distillation system of taking a cyclic carbonate and an aliphatic monohydric alcohol as starting materials, continuously feeding the starting materials into a continuous multi-stage distillation column in which a catalyst is present, carrying out reaction and distillation simultaneously in said column, continuously withdrawing a low boiling point reaction mixture containing the produced dialkyl carbonate from an upper portion of the column in a gaseous form, and continuously withdrawing a high boiling point reaction mixture containing the diol from a lower portion of the column in a liquid form, wherein:
(a) said continuous multi-stage distillation column comprises a tray column type distillation column having a cylindrical trunk portion having a length L (cm) and an inside diameter D (cm) and having a structure having thereinside an internals with a number of stages n, said internal being a tray having a plurality of holes, and further having a gas outlet having an inside diameter d₁ (cm) at a top of the column or in an upper portion of the column near to the top, a liquid outlet having an inside diameter d₂ (cm) at a bottom of the column or in a lower portion of the column near to the bottom, at least one first inlet provided in the upper portion and/or a middle portion of the column below said gas outlet, and at least one second inlet provided in the middle portion and/or the lower portion of the column above said liquid outlet, wherein:
(1) the length L (cm) satisfies the formula (1);
2100≦L≦8000 (1),
(2) the inside diameter D (cm) of the column satisfies the formula (2);
180≦D≦2000 (2),
(3) a ratio of the length L (cm) to the inside diameter D (cm) of the column satisfies the formula (3);
4≦L/D≦40 (3),
(4) the number of stages n satisfies the formula (4);
10≦n≦120 (4),
(5) a ratio of the inside diameter D (cm) of the column to the inside diameter d₁ (cm) of the gas outlet satisfies the formula (5)
3≦D/d₁≦20 (5), and
(6) a ratio of the inside diameter D (cm) of the column to the inside diameter d₂ (cm) of the liquid outlet satisfies the formula (6);
5≦D/d₂≦30 (6);
(b) the starting material cyclic carbonate is continuously introduced into said continuous multi-stage distillation column from at least one said first inlet provided between the 3^{rd} stage from the top of said continuous multi-stage distillation column and the (n/3)^{th} stage from the top of said continuous multi-stage distillation column;
(c) the starting material aliphatic monohydric alcohol contains in a range of from 1 to 15% by weight of the dialkyl carbonate; and
(d) the starting material aliphatic monohydric alcohol is continuously introduced into said continuous multi-stage distillation column from at least one said second inlet provided between the (n/3)^{th} stage from the top of said continuous multi-stage distillation column and the (2n/3)^{th} stage from the top of said continuous multi-stage distillation column.

2. The process according to claim 1, wherein a produced amount of the dialkyl carbonate is not less than 2 ton/hr.

3. The process according to claim 1 or 2, wherein a produced amount of the diol is not less than 1.3 ton/hr.

4. The process according to claim 1, wherein said d₁ and said d₂ satisfy the formula (7);
1≦d₁/d₂≦5 (7).

5. The process according to claim 1, wherein L, D, L/D, n, D/d₁, and D/d₂ for said continuous multi-stage distillation column satisfy respectively 2300≦L≦6000, 200≦D≦1000, 5≦L/D≦30, 30≦n≦100, 4≦D/d₁≦15, and 7≦D/d₂≦25.

6. The process according to claim 1, wherein L, D, L/D, n, D/d₁, and D/d₂ for said continuous multi-stage distillation column satisfy respectively 2500≦L≦5000, 210≦D≦800, 7≦L/D≦20, 40≦n≦90, 5≦D/d₁≦13, and 9≦D/d₂≦20.

7. The process according to claim 1, wherein the starting material aliphatic monohydric alcohol contains in a range of from 1.5 to 12% by weight of the dialkyl carbonate.

8. The process according to claim 7, wherein the starting material aliphatic monohydric alcohol contains in a range of from 2 to 10% by weight of the dialkyl carbonate.

9. The process according to claim 1, wherein the starting material aliphatic monohydric alcohol is continuously introduced into said continuous multi-stage distillation column from at least one inlet provided between the (2n/5)^{th} stage from the top of said continuous multi-stage distillation column and the (3n/5)^{th} stage from the top of said continuous multi-stage distillation column.

10. The process according to claim 1, wherein the tray is a sieve tray, said sieve tray having 100 to 1000 holes/m² in a sieve portion thereof.

11. The process according to claim 1, wherein the tray is a sieve tray, a cross-sectional area per hole of said sieve tray being in a range of from 0.5 to 5 cm².
